(19) United States
(12) Patent Application Publication
(10) Pub. No.: US 2003/0108599 A1
(43) Pub. Date: Jun. 12, 2003

(54) Novel capsule

(76) Inventors: Takahisa Takubo, Tokyo (JP); Hiroshi Onuki, Tokyo (JP); Eisaku Sai, Tokyo (JP); Kenji Miyata, Tokyo (JP)

Correspondence Address: Warner-Lambert Company, 201 Tabor Rd, Morris Plains, NJ, 07960, US

(21) Appl. No.: 10/288,624

(22) Filed: Nov. 5, 2002

(30) Foreign Application Priority Data
Nov. 9, 2001 (JP) 2001-344449

(51) Int. Cl. A61K 9/48

(52) U.S. Cl. 424/452

(57) ABSTRACT

A capsule including a physiologically acceptable nonionic surfactant coating. The coating provides excellent glide, anti-static properties and printing quality without preventing its degradation.

This application claims priority to Japanese Patent Application 2001-344449, field on Nov. 9, 2001.

### FIELD OF THE INVENTION

The present invention relates to a capsule for medicines or foods.

### BACKGROUND OF THE INVENTION

A wide variety of capsules are known such as soft capsules prepared by adding glycerin to gelatin, gelatin hard capsules which are essentially composed of gelatin and do not contain glycerin, cellulose-based hard capsules which are essentially composed of a cellulose derivative substrate, and the like. To produce these capsules, the capsules must have suitable glide in order to make the best use of the performance of a machine in inspection, printing, filling and packing steps. To provide this glide, some surface treatment must be made on the surface of each capsule under the present conditions.

Surface treating agents for the surface of a capsule are known. For example, magnesium stearate, starches, carnauba, talc and oily application agents such as vegetable oils are known surface treating agents. In the case of solid powders, there is a fault that it is difficult to control the application amount thereof. Further, in the case of solid powders, there is some fear of the health damage by inhaling scattered powders in the process of the surface treatment.

Since the oily application agents generally have a high dielectric constant and poor conductivity, a capsule coated therewith is readily charged with static electricity. Therefore, when a large number of capsules are handled, for example, they are set in a capsule filling machine and they may adhere to a capsule container such as a vinyl bag. The handling is thereby inconvenient. Further, when the surface of a capsule is printed with an ordinary capsule printing ink whose solvent is water or alcohol, there is a fault that the ink on the printed surface is readily rubbed off.

In order to solve the subject above, the object of the present invention is to provide a surface application agent for capsules, the amount of which is easily controlled, the application of which provides lubricity and an anti-static effect to a capsule and which has high affinity for a printing ink whose solvent is water or alcohol. Further, the uses of the capsule can be considered to be mainly such as a use for a medicine, a use for a food and a use for a medicine for animals; when it is used for a medicine or a food, the surface application agent must have safety to the human body when it is taken.

As a result of a wholehearted investigation in order to solve the above object, the inventors of the present invention have completed the present invention.

That is, the present invention relates to a capsule whose surface is coated with a physiologically acceptable nonionic surfactant.

The physiologically acceptable nonionic surfactants useful in the present invention is selected from cholesterol, sucrose fatty acid ester, stearyl alcohol, polyoxyl stearate 40, sorbitan sesquioleate, cetanol, cetomacrogol 1000, diethyl sebacate, sorbitan trioleate, polyoxyethylene octylphenyl ether, polyoxyethylene hardened castor oil 5, polyoxyethylene hardened castor oil 10, polyoxyethylene hardened castor oil 20, polyoxyethylene hardened castor oil 40, polyoxyethylene hardened castor oil 50, polyoxyethylene hardened castor oil 60, polyoxyethylene hardened castor oil 100, polyoxyethylene stearyl ether, polyoxyethylene sorbitol beeswax, polyoxyethylene (20) polyoxypropylene (20) glycol, polyoxyethylene (105) polyoxypropylene (5) glycol, polyoxyethylene (120) polyoxypropylene (40) glycol, polyoxyethylene (160) polyoxypropylene (30) glycol, polysorbate 20, polysorbate 60, polysorbate 65, polysorbate 80, macrogol 400, sorbitan monooleate, glycerin monostearate, sorbitan monostearate, sorbitan monolaurate, lauryl dimethylamine oxide solution, diethanolamide laurate, lauromacrogol and sorbitan monopalmitate, described in the Japanese Pharmacopoeia, medical Additive Provisions and Food Additive Provisions.

The physiologically acceptable nonionic surfactants preferable for the present invention is selected from cholesterol, polyoxyl stearate 40, sorbitan sesquioleate, cetomacrogol 1000, diethyl sebacate, sorbitan trioleate, polyoxyethylene octylphenyl ether, polyoxyethylene hardened castor oil 5, polyoxyethylene hardened castor oil 10, polyoxyethylene hardened castor oil 20, polyoxyethylene hardened castor oil 40, polyoxyethylene hardened castor oil 50, polyoxyethylene hardened castor oil 60, polyoxyethylene hardened castor oil 100, polyoxyethylene stearyl ether, polyoxyethylene sorbitol beeswax, polyoxyethylene (20) polyoxypropylene (20) glycol, polyoxyethylene (105) polyoxypropylene (5) glycol, polyoxyethylene (120) polyoxypropylene (40) glycol, polyoxyethylene (160) polyoxypropylene (30) glycol, polysorbate 20, polysorbate 60, polysorbate 65, polysorbate 80, macrogol 400, sorbitan monooleate, glycerin monosteareate, sorbitan monostearate, sorbitan monolaurate, lauryl dimethylamine oxide solution, diethanolamide laurate, lauromacrogol or sorbitan monopalmitate.

The physiologically acceptable nonionic surfactant more preferable for the present invention is selected from cholesterol, polyoxyl stearate 40, sorbitan sesquioleate, cetomacrogol 1000, sorbitan trioleate, polyoxyethylene hardened castor oil 60, polyoxyethylene (105) polyoxypropylene (5) glycol, polyoxyethylene (160) polyoxypropylene (30) glycol, polysorbate 20, polysorbate 60, polysorbate 80, macrogol 400, sorbitan monooleate, glycerin monostearate, sorbitan monostearate, sorbitan monolaurate, lauromacrogol or sorbitan monopalmitate.

The nonionic surfactant used in the present invention is preferably insoluble in water and soluble in an alcohol.

The nonionic surfactant which is insoluble in water and soluble in an alcohol is selected from cholesterol, sucrose fatty acid ester, stearyl alcohol, cetanol, cetomacrogol 1000, diethyl sebacate, sorbitan trioleate, polyoxyethylene octylphenyl ether, polyoxyethylene hardened castor oil 5, polyoxyethylene hardened castor oil 10, polyoxyethylene hardened castor oil 20, polyoxyethylene hardened castor oil 40, polyoxyethylene hardened castor oil 50, polyoxyethylene hardened castor oil 60, polyoxyethylene stearyl ether, polysorbate 65, sorbitan monooleate, sorbitan monostearate, sorbitan monolaurate, diethanolamide laurate, lauromacrogol and sorbitan monopalmitate; the preferable nonionic surfactant is selected from cholesterol, sucrose fatty acid ester, stearyl alcohol, cetanol, diethyl sebacate, sorbitan trioleate, polyoxyethylene hardened castor oil 5, polyoxyethylene hardened castor oil 10, polyoxyethylene hardened castor oil 20, polysorbate 65, sorbitan monooleate, sorbitan monostearate, sorbitan monolaurate or sorbitan monopalmitate, more preferably cholesterol, diethyl sebacate, sorbitan trioleate, polyoxyethylene hardened castor oil 5, polyoxyethylene hardened castor oil 10, polyoxyethylene hardened castor oil 20, polysorbate 65, sorbitan monooleate, sorbitan monostearate, sorbitan monolaurate or sorbitan monopalmitate.

In most of printing inks used in medicines, shellac which contains an alcohol as a solvent is used as a binder. Alcohol-soluble surfactants have good affinity for these printing inks.

A capsule essentially composed of gelatin or the like which is swollen with water is the mainstream whereas a surfactant which is insoluble in water does not give inconvenience such as swelling or deformation to the capsule, reduces friction and provides high lubricity to the capsule.

The nonionic surfactant used in the present invention preferably is a nonionic surfactant having an HLB value of 9 or less. The nonionic surfactant having an HLB of 9 or less can be selected from sorbitan trioleate (HLB=1.8), sorbitan monooleate (HLB=4.3), sorbitan monostearate (HLB=4.7), sorbitan monolaurate (HLB=8.6), cetanol, stearyl alcohol, sucrose fatty acid esters and sorbitan monopalmitate (HLB=6.7); as a preferable nonionic surfactant, sorbitan trioleate (HLB=1.8), sorbitan monooleate (HLB=4.3), sorbitan monostearate (HLB=4.7), sorbitan monopalmitate (HLB=6.7) and sorbitan monolaurate (HLB=8.6) can be mentioned. The most preferred nonionic surfactant in the present invention is sorbitan monolaurate, which is liquid at room temperature and does not give quality deterioration by rancidity.

The application amount of the nonionic surfactant is preferably 10 to 200 ppm, more preferably 10 to 100 ppm, much more preferably 30 to 100 ppm, the most preferably 50 to 100 ppm based on the weight of an empty capsule.

To carry out the present invention, the application amount of the nonionic surfactant can be controlled easily by being dissolved in or diluted with a solvent such as ethanol in a suitable ratio and spraying the resulting solution or being impregnated into a sponge and applied to the surface of a capsule in accordance with the conventional method.

The following examples are provided for the purpose of further illustrating the claimed. Although the effect of the invention is shown below concretely, the examples below do not limit the invention in any way.

### BRIEF DESCRIPTION OF THE DRAWINGS

FIG. 1 is a graph of the results of the glide test on capsules.

FIG. 2 is a graph of the results of the electrostatic property test on capsules.

### EXAMPLES

### Example 1

### Production of Capsule and Glide Test

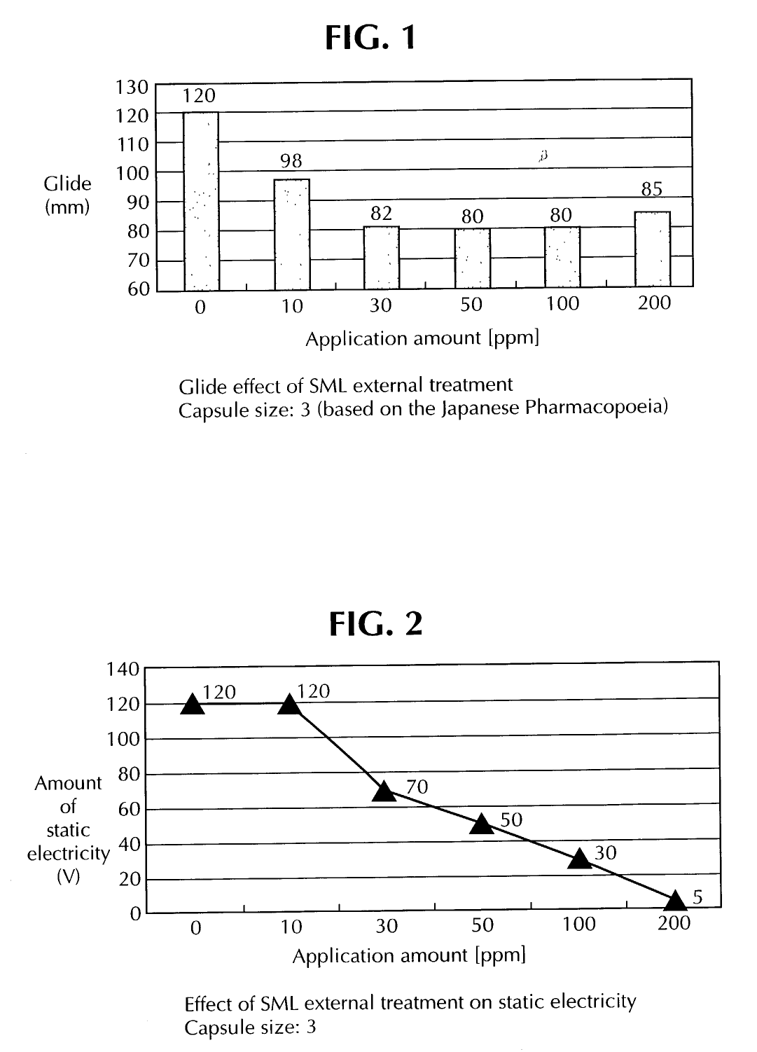

Commercially available sorbitan monolaurate (SML) was diluted with ethanol (EtOH) of the Japanese Pharmacopoeia in a weight ratio of 9:1 (SML: EtOH) and sprayed over empty hard gelatin capsules so that it adheres to the capsule surface with the amount of 10, 30, 50, 100 and 200 ppm based on the total weight of an empty hard gelatin capsule. After 30 minutes, a predetermined amount of the obtained capsules were placed in a transparent acryl box, followed by opening a shutter on one side of the box so that the capsules could slide out of the box by their own weight and the glide of the capsule was assayed by measuring the height of the capsules from the bottom when the flow of the capsules stopped. The better the glide is, the smaller the height of the capsules becomes. The results are shown in FIG. 1.

As shown in FIG. 1, the glide of the capsule is apparently improved as compared with that of uncoated capsules (the amount of adhesion: 0 ppm) by applying the application agent of the present invention to the outer surface of the capsule.

Electrostatic Property Test

Empty capsules coated with sorbitan monolaurate in the same manner as in Example 1 were placed in a stainless steel cup, which was then placed on an insulating sheet. The propeller of a stirrer was inserted into the stainless steel cup to stir the capsules at a predetermined revolution speed, and the electrostatic voltage of the capsules at 2 minutes after the start of stirring was measured by an electrostatic meter (SSD STATIRON-M2 of Shishido Electrostatic Ltd.). The results are shown in FIG. 2. It was confirmed that the amount of electrostatic charge was effectively reduced by the application agent of the present invention.

Printing Property Test 1

After the surface of an empty capsule applied with sorbitan monolaurate in the same manner as in Example 1 was printed with a black ink for medicines which is allowed to be used as a medicine (BLACK P-10 of Saneigen FFI Co., Ltd.), a Rub-off test where a piece of cellophane tape was affixed to and rubbed off from the printed surface was carried out. The results are shown in Table-1.

TABLE 1

| Surface | | |
|---|---|---|
| treating | amount of | |
| agent | application (ppm) | result of the Rub-off test |
| Sorbitan | 30 | satisfactory, no exfoliation of ink |
| monolaurate | 50 | satisfactory, no exfoliation of ink |
| | 200 | satisfactory, no exfoliation of ink |

Even when the application agent of the present invention was applied with 200 ppm based on the weight of an empty capsule, the exfoliation of the ink was not observed.

### Printing Property Test 2

After the surface of capsules was printed using a black ink and a blue ink for medicines (BLACK P-10 and BLUE B2-30 of Saneigen FFI Co., Ltd.) and a gray ink (Gray S9-27617 of Calcon Co., Ltd.) in the same manner as the printing property test 1, 20,000 capsules for each ink were inspected the printing quality based on the equivalent inspection standard as that for in-house shipping products by the capsule appearance inspection machine made by the applicant. The results are shown in Table 2.

TABLE 2
Number of capsules from which ink was exfoliated/number of samples:
20,000 capsules each

| Ink | Defect | SML 30 ppm |
|---|---|---|
| Gray ink | Slightly Unsatisfactory | 0 |
| | Slightly Defective | 0 |
| Black ink | Slightly Unsatisfactory | 0 |
| | Slightly Defective | 0 |
| Blue ink | Slightly Unsatisfactory | 0 |
| | Slightly Defective | 0 |

As shown in the table, no failure was observed in the capsule of the present invention, the sufficient practicability for the printing quality has been confirmed.

What is claimed is:

1. A capsule comprising a surface coating including a physiologically acceptable nonionic surfactant.

2. The capsule according to claim 1, wherein said nonionic surfactant is insoluble in water and soluble in an alcohol.

3. The capsule according to claim 1, wherein said nonionic surfactant has an HLB value of 9 or less.

4. The capsule according to claim 1, wherein the coating weight of the nonionic surfactant is 10 to 200 ppm based on the weight of an empty capsule.

5. The capsule according to claim 1, wherein said nonionic surfactant is selected from the group consisting of sorbitan trioleate, sorbitan monooleate, sorbitan monostearate, sorbitan monolaurate, stearyl alcohol, cane sugar aliphatic acid ester, sorbitan monopalmitate and mixtures thereof.

6. The capsule according to claim 5, wherein said nonionic surfactant is selected from the group consisting of sorbitan trioleate, sorbitan monooleate, sorbitan monostearate, sorbitan monolaurate, sorbitan monopalmitate and mixtures thereof.

7. The capsule according to claim 6, wherein said nonionic surfactant is sorbitan monolaurate.

8. The capsule according to claim 2, wherein the coating weight of the nonionic surfactant is 10 to 200 ppm based on the weight of an empty capsule.

9. The capsule according to claim 2, wherein said nonionic surfactant is selected from the group consisting of sorbitan trioleate, sorbitan monooleate, sorbitan monostearate, sorbitan monolaurate, stearyl alcohol, cane sugar aliphatic acid ester, sorbitan monopalmitate and mixtures thereof.

10. The capsule according to claim 9, wherein said nonionic surfactant is selected from the group consisting of sorbitan trioleate, sorbitan monooleate, sorbitan monostearate, sorbitan monolaurate, sorbitan monopalmitate and mixtures thereof.

11. The capsule according to claim 10, wherein said nonionic surfactant is sorbitan monolaurate.

12. The capsule according to claim 3, wherein the coating weight of the nonionic surfactant is 10 to 200 ppm based on the weight of an empty capsule.

13. The capsule according to claim 3, wherein said nonionic surfactant is selected from the group consisting of sorbitan trioleate, sorbitan monooleate, sorbitan monostearate, sorbitan monolaurate, stearyl alcohol, cane sugar aliphatic acid ester, sorbitan monopalmitate and mixtures thereof.

14. The capsule according to claim 13, wherein said nonionic surfactant is selected from the group consisting of sorbitan trioleate, sorbitan monooleate, sorbitan monostearate, sorbitan monolaurate, sorbitan monopalmitate and mixtures thereof.

15. The capsule according to claim 14, wherein said nonionic surfactant is sorbitan monolaurate.

16. The capsule according to claim 4, wherein said nonionic surfactant is selected from the group consisting of sorbitan trioleate, sorbitan monooleate, sorbitan monostearate, sorbitan monolaurate, stearyl alcohol, cane sugar aliphatic acid ester, sorbitan monopalmitate and mixtures thereof.

17. The capsule according to claim 16, wherein said nonionic surfactant is selected from the group consisting of sorbitan trioleate, sorbitan monooleate, sorbitan monostearate, sorbitan monolaurate, sorbitan monopalmitate and mixtures thereof.

18. The capsule according to claim 17, wherein said nonionic surfactant is sorbitan monolaurate.
